(19) United States
(12) Patent Application Publication
(10) Pub. No.: US 2018/0085204 A1
(43) Pub. Date: Mar. 29, 2018

(54) DENTAL FLOSSING BAND

(71) Applicant: CRISTINA MIKLAS, SOAVE (IT)

(72) Inventor: CRISTINA MIKLAS, SOAVE (IT)

(21) Appl. No.: 15/718,360

(22) Filed: Sep. 28, 2017

Related U.S. Application Data
(60) Provisional application No. 62/401,455, filed on Sep. 29, 2016.

(51) Int. Cl. A61C 15/04; A61K 8/21; A61K 8/19; A61K 8/34; A61K 8/97; A61K 8/92; A61Q 11/00

(52) U.S. Cl. CPC A61C 15/041; A61K 8/21; A61K 8/19; A61Q 11/00; A61K 8/97; A61K 8/922; A61K 8/34

(57) ABSTRACT

A flexible and non extendable band for flossing teeth, which can be made in various materials, sizes and proportions, that may have various features, and that may be coated or soaked in various substances. It is used between teeth and under the gum line to clean such areas effectively and easily.

### CROSS REFERENCE TO RELATED APPLICATION

This application claims the benefit of the prior-filed U.S. Provisional Application No. 62401455, filed on Sep. 29, 2016 by Cristina Miklas.

### BACKGROUND OF THE INVENTION

### Field of Invention

The present invention is in the field of oral care. More specifically the present invention is in the field of dental hygiene. More specifically the present invention is in the field of food and plaque removal from teeth.

### Description Of The Related Art

Dental professionals recommend the removal of dental plaque at least on a daily basis. Regular brushing is not sufficient for the removal of plaque between teeth and under the gum line. Flossing is highly recommended to clean these places and to avoid plaque buildup and bacterial growth that can lead to cavities, tooth decay, gum disease and bad breath.

Many flossing devices have been invented yet they have drawbacks.

Conventional dental floss is generally not comfortable, practical, and fast to use and therefore may not encourage the continuation of such important routine. Conventional floss requires selecting the right length of floss, than wrapping the floss around the fingers and then re-wrapping it repeatedly during the cleaning session such that even a few seconds that must be invested daily can weigh heavily on a person behavior. Additionally, the floss often becomes too tight around the fingers around which they are wrapped causing pain and marks. Moreover, children in particular find it hard to properly wrap the floss around their fingers and keep it in that position, such that they do not floss properly with it. Similar problems may affect people with as arthritis and impaired hand mobility. Finally, such item produces unnecessary waste because the vast majority of the floss (and of its raw materials and eventual active ingredients) is not really used for flossing but rather for wrapping the floss around the fingers.

Most dental flossing tools (such as flossing picks and many flossing devices with handles just to mention a few) do not give the control needed to stop the floss from hitting the gums after they pass through a tight (very narrow) spot between two teeth, thus causing pain and even bleeding of the gums.

All dental flossing tools in which the floss is pulled in a straight line lack maneuverability: when using them, it is much harder or impossible to surround the round base of the tooth. This impedes the proper and complete cleaning of the base of the tooth especially under the gum line.

Some dental flossing tools or dental floss holders require additional assembling steps and preparation time which may discourage establishing and/or maintaining a daily flossing routine with those individual with less time available or who are less willing to floss.

Most dental flossing tools or dental floss holders require handles and additional elements which increase the production costs and the final product cost. This may discourage the purchase of such item (and the flossing routine) in lower income families.

Most dental flossing tools and dental floss holders require handles and additional elements increasing use of plastics and other materials and the amount of waste produced. This may discourage the purchase of such item (and the flossing routine) in the more environmentally conscious individuals.

The above described drawbacks, in different degrees, may lead to reducing and the effectiveness of flossing and/or the frequency of flossing in even the more dedicated people. They even may discourage some people from flossing all together.

Even after many efforts in this field, there is still the need for a flossing device that is effective and yet easy, comfortable, and faster to use, economic to produce and to purchase, not wasteful, and even environmental friendly and more adaptable to the user's needs. Such features would help increasing the commitment to flossing, its proper execution, and its effectiveness.

### SUMMARY OF THE INVENTION

The present invention is a flexible and non extendable band for flossing teeth, which can be made in various materials, sizes and proportions, that may have various features, and that may be coated or soaked in various substances.

It is one particular object of the invention disclosed herein, to provide an item for flossing that is effective and yet easier, more comfortable, faster to use, more economic to produce and to purchase, less wasteful, and in some embodiments also more environmental friendly, more adaptable to an user's need, and/or more appealing to children than what is already available.

It is a further object of this invention to provide a flossing device that increases the commitment to flossing, its proper execution, and its effectiveness.

The invention described in here achieves the goals described above and avoids the problems or solves the shortcomings of prior art.

### BRIEF DESCRIPTION OF THE DRAWINGS

FIG. 1A is a perspective view of a preferred embodiment of a dental flossing band according to the present invention;

FIG. 1B is a side view of the dental flossing band of FIG. 1A;

FIG. 1C is a top view of the dental flossing band of FIG. 1A;

FIG. 1D is a view of the dental flossing band of FIG. 1A being used;

FIG. 2A is a perspective view of another preferred embodiment of a dental flossing band according to the present invention;

FIG. 2B is a side view of the dental flossing band of FIG. 2A;

FIG. 2C is a top view of the dental flossing band of FIG. 2A;

FIG. 3A is a perspective view of another preferred embodiment of a dental flossing band according to the present invention;

FIG. 3B is a side view of the dental flossing band of FIG. 3A;

FIG. 3C is a top view of the dental flossing band of FIG. 3A;

### DETAILED DESCRIPTION OF THE INVENTION

The terminology used herein is for the purpose of describing particular embodiments and is not intended to be limiting of the invention. As used herein, the terms “and/or” includes any and all combinations of one or more of the listed items.

The present disclosure is to be considered an exemplification of the invention; indeed this invention may be embodied in many different forms and should not be construed as limited to the embodiments set forth herein.

Some embodiments are described in reference to the accompanying drawings but not all the embodiments and optional features of the inventions are shown in the figures. It is not intended to limit the invention to the specific embodiments illustrated by the figures.

Certain embodiments of the present invention include, but are not limited to the ones described below.

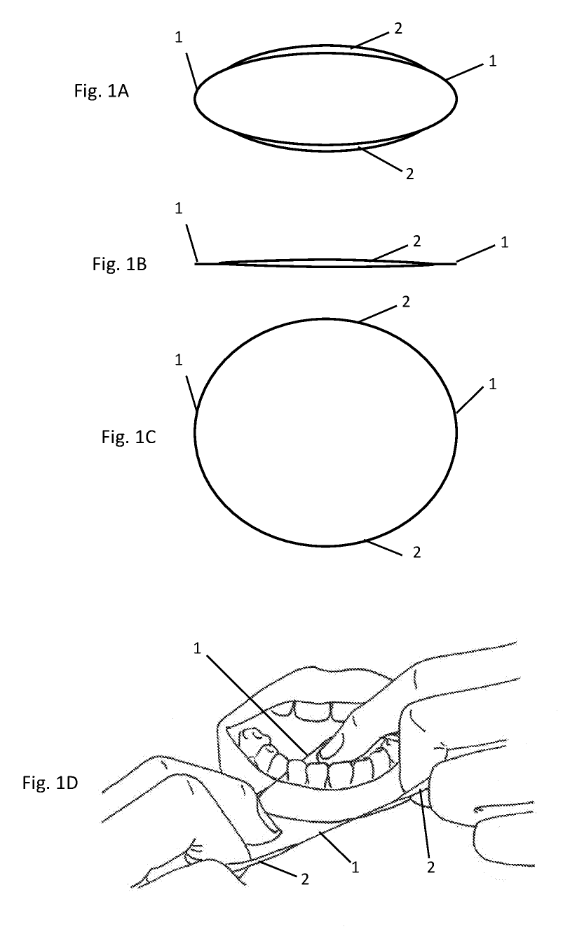

Referring now to FIGS. 1A, 1B, 1C and 1D, there is shown a flexible dental flossing band with two opposing thin segments 1 and other two opposing segments 2 that are as thin as segments 1 where such segments 1 and 2 join, yet which are thicker, in a direction lying perpendicular to the plane occupied by the flossing band as shown by FIGS. 1B and 1C, at regions distal from the joining points between the two segments 1 and 2. The thickness of segments 2 decreases smoothly from the central point of the segments 2 until they reach at both ends the thickness of segments 1. The segments 2 and the segments 1 are alternated and seamlessly connected forming a closed circular band.

In more detail, still referring to the invention of FIG. 1A, 1B, 1C, and 1D, during the flossing of the teeth the flossing band is held with both hands normally by the user with the middle, ring and/or pinky fingers. While the middle, ring and/or pinky fingers hold the band by the segments 2 with a sufficient tension, the index fingers and/or the thumb fingers gently drive or maneuvers the segment 1, so that it slides between the teeth and under the gum lines as shown in FIG. 1D. Typically, to remove food debris and plaque in this area, the user may slide the segment 1 between two teeth than move it carefully up and down, then he/she may curve segment 1 around the tooth and let it dip below the gum line (preferably, about 2-3 millimeters down). Additionally, the user may move the band in a back-and-forth motion to help scrape, still using one of the sections 1, additional plaque and debris. When the user is done cleaning an area, he/she may gently extract the band back out the way it came. One or both segments 1 may be used while repeating the process for all areas to be cleaned.

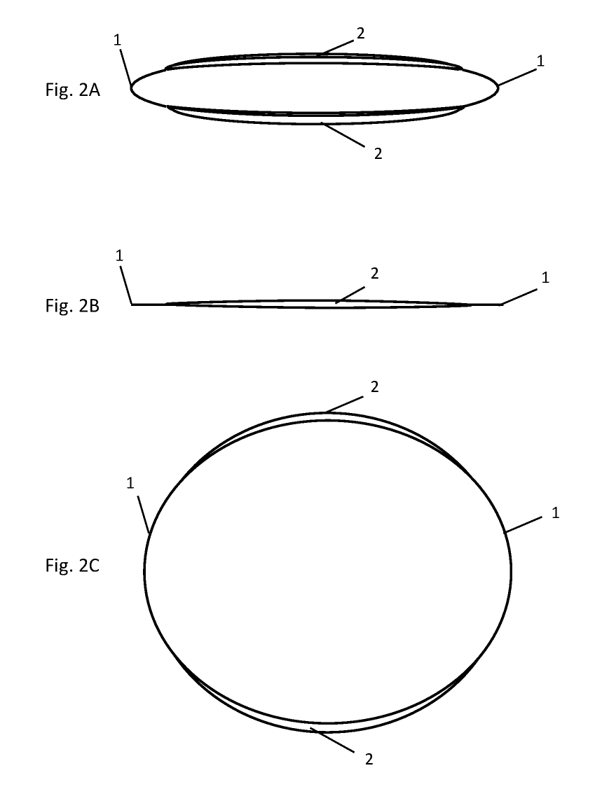

In one additional embodiment of the invention, shown in FIG. 2A, 2B, 2C, two opposing sections 2 are thicker than the segments 1, in two mutually perpendicular directions, one perpendicular to the plane occupied by the flossing band and one in the plane occupied by the flossing band, as shown in FIGS. 2B and 2C. The width of segments 2 may range from 0.01 mm to 10 mm, however the most preferred width may range from 0.1 mm to 3 mm. The sections 2 which are the thicker-parts of the band, may be used to floss areas between teeth with larger gaps or simply to provide more comfort during the regular use described above.

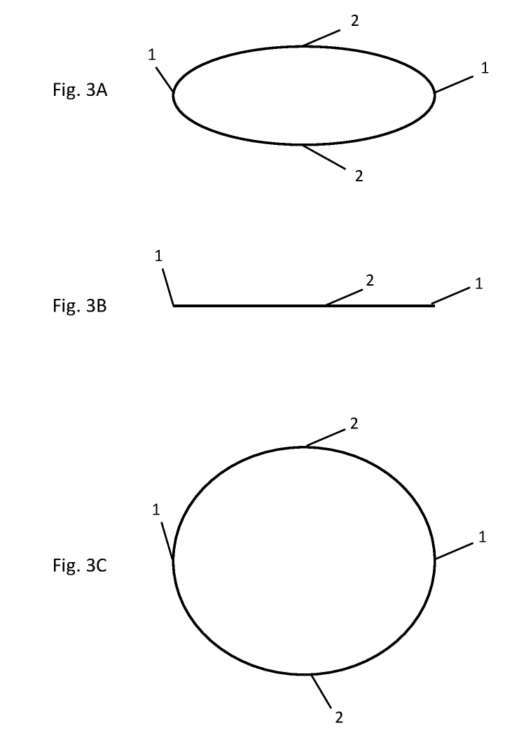

In one additional embodiment of the invention the whole band has only one shape, with the same thickness all around. FIG. 3A, 3B, 3C depicts this embodiment in the case of a minimal sectional size. This is the simpler embodiment of the invention.

In more detail, the flossing band may be manufactured with various non-stretchable materials or compositions of such materials including, but not limited to: Nylon, Polymer, Polytetrafluoroethylene, Polyester and/or silk.

In more detail, the flossing band may be produced in various lengths (or circumferences). The length should be sufficient for proper handling. Most children and adults would find a length between 15 cm to 25 cm to be ideal (equivalent to approximately a diameter from 4.77 cm to 7.96 cm). The length of segments 1 and 2 may vary. The FIG. 1A, 1B, 1C, 2A, 2B, 2C for example, show the case of four segments of equal length where each segment is ¼ of the total length of the band. However, segments 1 may also be longer or shorter then segments 2.

In more detail, the invention in FIG. 1A, 1B, 1C and the embodiment in FIG. 2A, 2B, 2C, The thickness of segments 2 can be manufactured in various sizes however the most suitable for the described use is from 1 mm to 10 mm.

Additionally, the segments 2 of the embodiment in FIG. 1A, 1B, 1C and the segments 2 of the embodiment in FIG. 2A, 2B, 2C may be manufactured with bonded abrasive material or may be coated on the external side with abrasive materials. Such sections of the invention may then be used to scrape off the plaque buildup that has already hardened and therefore is more resistant. The abrasive particles may be natural and/or manufactured such as: Arkansas Stone, Chalk, Corundum, Emery, Garnet, Pumice, Quartz, Sand, Tripoli, Zirconium silicate, Cuttle, Kieselguhr, Silicon carbide, Aluminium oxide, Synthetic diamond, Rouge, or Tin oxide). Abrasive particles may have different sizes such as: Coarse, Medium coarse, Medium, Fine, and/or Superfine.

Additionally, the segments 1 of the invention in FIG. 1A, 1B, 1C, the segments 1 of the embodiment in FIG. 2A, 2B, 2C and the embodiment in FIG. 3A, 3B, 3C may be produced in various thickness and shape to match users' needs and preferences. One or both segments 1 may be: round and very fine (less than 1 mm), thick (more than 1 mm), flattened (ribbon like), spongy and/or soft.

In more detail, the segments 1 and/or segments 2 of the flossing band in FIG. 1A, 1B, 1C, the segments 1 and/or 2 of the embodiment in FIG. 2A, 2B, 2C and the embodiment in FIG. 3A, 3B, 3C can be coated with a wax components, including but not limited to: Synthetic wax, vegetable wax, and/or bee wax. Such coating renders the band easier to slide between teeth. In more detail, the segments 1 and/or segments 2 of the flossing band in FIG. 1A, 1B, 1C, the segments 1 and/or 2 of the embodiment in FIG. 2A, 2B, 2C and the embodiment in FIG. 3A, 3B, 3C can be soaked in, or coated with active ingredients, including but not limiting: cooling, whitening, antibacterial, disinfecting, usage indication, herbal oil or extracts, and/or flavoring ingredients. Examples may be: floride, baking soda, alcohol, herbal extracts (such as Sage, Echinacea, Chamomilla, Mentha, Thyme, Eucalypt extracts), herbal oils (such as Sage, Echinacea, Chamomilla, Mentha, Thyme, Eucalypt oil). Also, the segments may be coated with any additional useful ingredient suitable for oral care and/or appealing to users. The materials used for soaking or coating are deposited on the teeth and the gums during usage releasing their benefits during and after the flossing sessions.

The invention and all its embodiments can be manufactured in different colors especially in order to appeal to children and to encourage them to use it and therefore to floss (Which color will come out of the box tonight?).

In one additional version of all the embodiments described above the band is reusable because it is manufactured with semi resistant and washable materials. It may be rinsed and reused a few times and then be disposed of when it is sufficiently worn out.

In one additional version of all the embodiments described above the band is reusable and durable because it is manufactured with durable and washable materials.

The advantages of the present invention include, without limitation that, compared to conventional dental floss and/or other items previously invented for flossing, it is more effective, easier to use, more comfortable to hold and maneuver, faster to use, more economic to produce and to purchase, less wasteful, and in some embodiments also more environmentally friendly, more adaptable to the user's needs, and/or more appealing to children.

The flossing band and its embodiments described herein achieve the objectives described above and avoids or solves the shortcomings of prior art. In fact:
It is fast and easy to use because it does not need to be cut, prepared, assembled, or wrapped round the fingers as required by many others dental flossing items.
It is more comfortable than regular dental floss because it is not wrapped around the fingers, which results in strangulation of the fingers themselves (causing soreness and leaving marks).
It is possible to orient it to reach specific places in the mouth, to wrap and surround each side of the tooth and drive it below the gum line. Therefore it is adapted to clean most efficiently and effectively the tooth in the places not reached by the tooth brush. Such important movement is not possible with all the prior art items that keep the floss in a rigid straight line.
It is easier to control and stop it before it hits the gums after passing through a tight spot between teeth, because the leverage point (index and/or thumb fingers) is near the gums and not at the end of a tool or handle.
It is less wasteful compared to the traditional floss because it is a flossing band that does not require additional length to be wrapped around the fingers (approximately only 1/10 of the traditional flossing treads are in contact with the teeth and therefore cleaning them despite having all the active ingredients and components for that purpose).
It is less wasteful compared to other items because it is a flossing band that does not require additional parts, handles, bases and mechanisms to function.
It is more adaptable to one's needs especially in the different embodiments and combinations described above.
It is more appealing to children because it is easier to use, especially in the versions that are colored and/or soaked or coated with children-appealing flavors (such as Cherry, buddle gum for example).

While materials and sizes and features have been described, the invention is not limited to those described. The present invention may be constructed with other materials, in other sizes, with other features, and with other coating substances.

In a broad embodiment, the invention is a band for flossing the teeth that is non extendable yet flexible, that can be made in various materials, sizes and proportions, that may have various features, and that may be coated or soaked with various substances.

While the present invention has been described in terms of particular embodiments and applications, in both summarized and detailed forms, it is not intended that these descriptions in any way limit its scope to any such embodiments and applications, and it will be understood that many substitutions, changes and variations in the described embodiments, applications and details of the method and system illustrated herein and of their operation can be made by those skilled in the art without departing from the spirit of this invention.

1. A dental flossing band comprising:
two opposing first segments; and
two opposing second segments;
said first segments being joined to said second segments at respective joining points such that said dental flossing band has a shape of a closed loop.

2. The dental flossing band according to claim 1, wherein said second segments are as thin as said first segments at said joining points, and said second segments are thicker than said first segments at regions of said second segments arranged distally from said joining points.

3. The dental flossing band according to claim 2, wherein said second segments are thicker than said first segments at said regions of said second segments arranged distally from said joining points in a direction that is perpendicular to a plane of arrangement of said flexible flossing band.

4. The dental flossing band according to claim 2, wherein said second segments decrease in thickness from a central point of said second segments towards said joining points.

5. The dental flossing band according to claim 2, wherein said second segments are thicker than said first segments at said regions of said second segments arranged distally from said joining points in two mutually perpendicular directions including a first direction that is perpendicular to a plane of arrangement of said flexible flossing band and a second direction that lies in said plane of arrangement.

6. The dental flossing band according to claim 5, wherein said second segments have a width between 0.01 mm to 10 mm.

7. The dental flossing band according to claim 5, wherein said second segments have a width between 0.1 mm to 3 mm.

8. The dental flossing band according to claim 1, wherein said dental flossing band has a same thickness all around said a closed loop.

9. The dental flossing band according to claim 1, wherein said dental flossing band is non-extendable and made of non-stretchable materials.

10. The dental flossing band according to claim 9, wherein said non-stretchable materials are selected from the group comprising: nylon, polymer, polytetrafluoroethylene, polyester and silk.

11. The dental flossing band according to claim 1, wherein said dental flossing band has a length between 15 cm to 25.

12. The dental flossing band according to claim 1, wherein said first segments and said second segments are all of equal length where each segment is one quarter of a total length of the dental flossing band.

13. The dental flossing band according to claim 1, wherein said first segments are longer or shorter then said second segments.

14. The dental flossing band according to claim 1, wherein said second segments 2 are made with bonded abrasive material or may be coated on an external side with abrasive materials.

15. The dental flossing band according to claim 14, wherein said abrasive materials are selected from the group comprising: Arkansas Stone, Chalk, Corundum, Emery, Garnet, Pumice, Quartz, Sand, Tripoli, Zirconium silicate, Cuttle, Kieselguhr, Silicon carbide, Aluminium oxide, Synthetic diamond, Rouge, and Tin oxide.

16. The dental flossing band according to claim 1, wherein said first segments and/or said second segments are coated with wax components selected from the group comprising: synthetic wax, vegetable wax, and bee wax.

17. The dental flossing band according to claim 1, wherein said first segments and/or said second segments are soaked in, or coated with active ingredients selected from the group comprising: cooling, whitening, antibacterial, disinfecting, usage indication, herbal oil or extracts, and flavoring ingredients.

18. The dental flossing band according to claim 17, wherein said active ingredients are selected from the group comprising: floride, baking soda, alcohol, herbal extracts, and herbal oils.

19. The dental flossing band according to claim 1, wherein said dental flossing band has a selected color.

20. The dental flossing band according to claim 1, wherein said dental flossing band is made of durable and washable materials.
